(19) United States
(12) Patent Application Publication
(10) Pub. No.: US 2024/0242585 A1
(43) Pub. Date: Jul. 18, 2024

(54) SYSTEM AND METHOD FOR MONITORING GAS ACCUMULATION IN ENCLOSED SPACES

(71) Applicant: SAUDI ARABIAN OIL COMPANY, Dhahran (SA)

(72) Inventors: Sultan M. AL-QAHTANI, Dammam (SA); Khaled M. AL-MUAIWEED, Al Hassa (SA); Mohammed S. AL-QAHTANI, Dammam (SA)

(73) Assignee: SAUDI ARABIAN OIL COMPANY, Dhahran (SA)

(21) Appl. No.: 18/154,541

(22) Filed: Jan. 13, 2023

(51) Int. Cl. G08B 21/12; G01N 33/00; G08B 31/00

(52) U.S. Cl. CPC G08B 21/12; G01N 33/0031; G01N 33/0063; G01N 33/0075; G08B 31/00

(57) ABSTRACT

A system includes a gas accumulation sensor installed within an enclosed space, a communications transceiver installed at an entrance to the enclosed space communicatively coupled to the gas accumulation sensor, and a cloud-based platform storing a gas accumulation application. The gas accumulation application receives one or more readings from the gas accumulation sensor via the communications transceiver, processes the one or more readings from the gas accumulation sensor, and alerts an operator with a safety status of the enclosed space.

### FIELD OF THE DISCLOSURE

The present disclosure relates generally to digitizing safety testing processes and, more particularly, to the detection and reporting of unsafe working conditions in enclosed spaces.

### BACKGROUND OF THE DISCLOSURE

When entering an enclosed space in which toxic gases may accumulate, safety protocols require the testing of gas accumulation within the space to determine if the space is safe for an operator to enter. Current practices involve the opening of the enclosed space, such as a telecommunications maintenance hole, and taking a reading near the entrance to the enclosed space to determine the accumulated gas levels within. The majority of the accumulated gas, however, may remain at or near the bottom of the enclosed space, particularly in vertical shafts or holes. As such, the acquisition of readings near the surface or entrance may yield results which are not representative of the gas levels throughout the enclosed space. In some instances, enclosed spaces deemed safe based on current safety practices may expose operators to unsafe levels deeper within the enclosed space. Further, high levels of accumulated gas within enclosed spaces may expose operators to toxic gases while opening the enclosed space, with no internal detection mechanism to warn of these high levels.

Accordingly, a system which may constantly monitor gas accumulation within an enclosed space and communicate the gas accumulation status with an external operator is desirable.

### SUMMARY OF THE DISCLOSURE

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

According to an embodiment consistent with the present disclosure, a system includes a plurality of gas accumulation sensors each operable to detect a gas within an associated enclosed space, a communications transceiver communicatively coupled to each gas accumulation sensor, and a computer platform operable to execute a gas accumulation application. The gas accumulation application receives one or more readings from each gas accumulation sensor via a communicatively coupled communications transceiver, processes the one or more readings, and alerts an operator with a safety status of the enclosed space.

In a further embodiment, a method includes receiving one or more measurements relating to gas in one or more enclosed spaces from sensors that are each associated with an enclosed space, determining a status of each enclosed space including whether hazardous conditions exist from the one or more measurements, and alerting an operator to the status of each enclosed space. The one or more measurements include a level of accumulated toxic gas, a presence of flammable gas, an oxygen content reading, or any combination thereof.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1 is a block diagram representation of a system with an IoT-enabled gas accumulation sensor installed within an enclosed space.

FIG. 2 is an example of a method for receiving sensor data and assessing a status of an enclosed space.

FIG. 3 is an example of a computer system that can be employed to execute one or more embodiments of the present disclosure.

### DETAILED DESCRIPTION

Embodiments of the present disclosure will now be described in detail with reference to the accompanying Figures. Like elements in the various figures may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

Embodiments in accordance with the present disclosure generally relate to digitizing safety testing processes and, more particularly, to the detection and reporting of unsafe working conditions in enclosed spaces. The placement of a sensor near the bottom of an enclosed space may enable more accurate readings of the safety conditions within the enclosed space for operator interaction. The communication between the sensor and the Internet of Things (IoT) may enable a cloud-based platform to monitor conditions within the enclosed space remotely, and may obviate the need to open a potentially unsafe enclosed space for testing. The smart operations enabled by the embodiments disclosed herein may enhance the safety and productivity of maintenance operations within enclosed spaces.

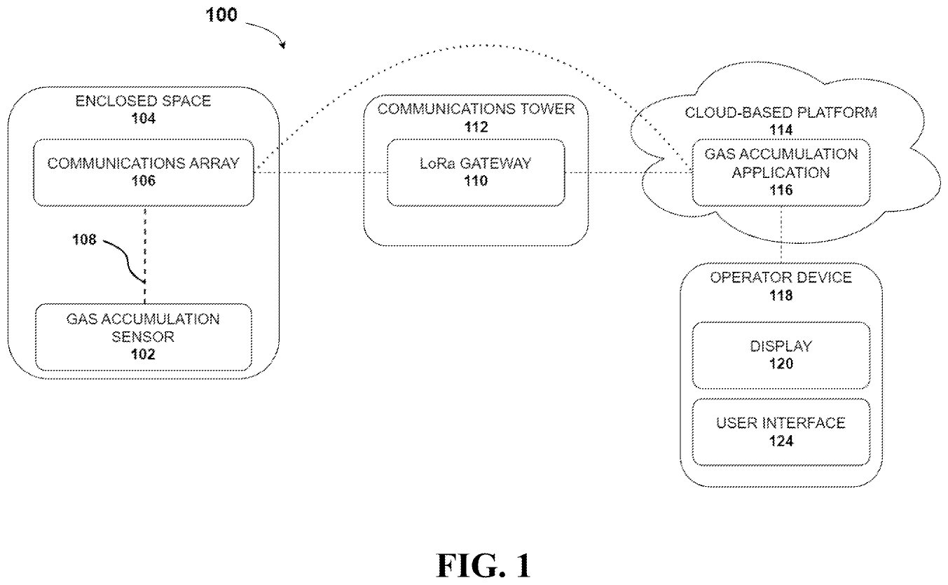

FIG. 1 is a block diagram representation of a system 100 with an IoT-enabled gas accumulation sensor 102 installed within an enclosed space 104, such as a telecommunications maintenance hole. The gas accumulation sensor 102 (hereinafter “the sensor 102”) may be installed within a lower-end of the enclosed space 104, such that the measurements output by the sensor 102 may capture the environmental conditions near the bottom of the enclosed space as some gases of concern may be heavier than air and may settle in the bottom. In at least one embodiment, the sensor 102 may be installed about 0.5 meters from the bottom, or ground level, of the enclosed space 104. The inclusion of the sensor 102 in the lower-end of the enclosed space 104 may provide increased safety compared to standard practices of measurements inside an entryway of the enclosed space 104, as the harmful or flammable gas may settle and accumulate near the bottom of the enclosed space 104 such that standard practices may fail to capture accurate readings. The sensor 102 may monitor for the accumulation of toxic or flammable gas within the enclosed space 104, and may further test for the oxygen content within the enclosed space 104, such that the sensor 102 covers all required safety checks for work in enclosed spaces (e.g. OSHA standard 1910.146(c)(5)(ii)(C)). The sensor 102, therefore, may represent a single sensor or measurement device, or a plurality of sensors or measurement devices in an assembly that may be installed within the enclosed space 104 without departing from the scope of this disclosure.

The sensor 102 may be installed within the lower-end of the enclosed space, while a communications transceiver 106 may be installed at or near the entry to the enclosed space 104, such that the communications transceiver 106 may send and receive signals without interference, for example by being obstructed by the structure defining the enclosed space. As such, the sensor 102 and the communications transceiver 106 may be communicatively coupled via a wire 108, which may be a low-voltage cable for the transmission of readings from the sensor 102 to the communications transceiver 106. In some embodiments, the wire 108 may provide power to the gas accumulation sensor 102 from a power source within or near the communications transceiver 106. In further embodiments, the sensor 102 includes a wireless communication component which enables transmission to the communications transceiver 106 without the wire 108.

The communications transceiver 106 may be installed at or near the entry to the enclosed space 104 in order to properly communicate with an intermediate communications system, directly with the internet, or with an independent network forming an IoT. In some embodiments, a LoRa gateway 110 may be within range of the communications transceiver 106, such that the sensor 102 may be in communication with additional devices or servers using a long-range, low-power radio frequency. In at least one embodiment, the LoRa gateway 110 is installed within an existing communications tower 112, such that the power and transmission infrastructure is utilized by the LoRa gateway 110 without the need for additional construction. The LoRa gateway 110, as well as the communications transceiver 106, may enable communication between the sensor 102 and a cloud-based platform 114 which contains or supports a gas accumulation application 116. In some embodiments, however, the communications transceiver 106 may directly or indirectly access the cloud-based platform 114 without the need for the LoRa gateway 110.

The gas accumulation application 116 (hereinafter “the application 116”) on the cloud-based platform 114 may enable the collection, processing, and tracking of data received from the sensor 102 within the enclosed space. The application 116 may receive one or more readings from the sensor 102, via the communications transceiver 106 or the LoRa gateway 110, and may store the readings on a cloud server or other remote device within the cloud-based platform 114. The readings from the sensor 102 may be processed by the application 116 to determine if the conditions within the enclosed space 104 are hazardous based upon predetermined criteria. Further, the sampling rate of the sensor 102 may determine the function of the application 116. In some embodiments, real-time/report-by-exception monitoring of the enclosed space 104 may trigger an alert at any time within the application 116 upon processing the readings. In alternate embodiments, the sensor 102 may monitor the enclosed space 104 with a periodic cycle (e.g., sampling every five minutes) such that the application 116 must wait for the periodic reading in order to determine the conditions within the enclosed space 104.

Once receiving the data within the cloud-based platform 114, the application 116 may determine a real-time status of the enclosed space 104. Further, with an accumulated history of data from the sensor 102, the application 116 may further generate historical trends which may include evaluations of the enclosed space 104 over time. From the data translated into the time-domain, the application 116 may perform further analyses which may show the frequency and speed of hazard events within the enclosed space 104. The application 116 may provide insight into the operation of one or more enclosed spaces 104, such that the most historically hazardous enclosed spaces 104 may be flagged for maintenance, review, or quality control to prevent further hazardous events. The insights provided by the application 116 may include heatmaps of individual enclosed spaces 104, or of all enclosed spaces 104 in a geographic area, to provide a view of the overall conditions within a desired area.

Following the data collection and processing, the application 116 may provide individual readings, tailored results, or a full report of operations to an operator device 118. The operator device 118 may include at least a display 120 which enables an operator to view conditions within the enclosed space 104, receive alerts regarding hazardous conditions within the enclosed space 104, or to view the most problematic enclosed spaces for preventative maintenance. The display 120 on the operator device 118 may display a business intelligence dashboard for the collection and display of data from the application 116.

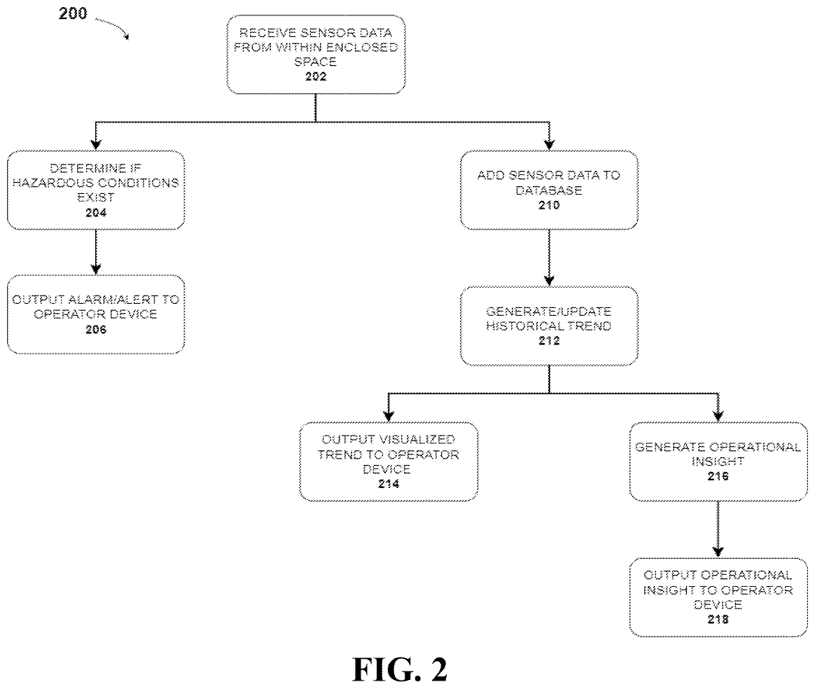

In view of the structural and functional features described above, example methods will be better appreciated with reference to FIG. 1. While, for purposes of simplicity of explanation, the example method of FIG. 2 is shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement the method, and conversely, some actions may be performed that are omitted from the description.

FIG. 2 is an example of a method 200 for receiving sensor data and assessing a status of an enclosed space (e.g., the enclosed space 104). The method 200 may be implemented by the system 100, as shown in FIG. 1. Thus, reference can be made to the example of FIG. 1 in the example of FIG. 2. The method 200 may begin at 202 with receipt of the sensor data provided by the sensor (e.g., the sensor 102) from within the enclosed space. As previously discussed, the sensor may be located near the bottom of the enclosed space to provide measurements in the most hazardous area of the enclosed space. The sensor data may be transmitted via a communications transceiver (e.g., the communications transceiver 106) and may pass through a LoRa gateway (e.g., the LoRa gateway 110).

The sensor data received at 202 may be immediately processed at 204 in order to determine if hazardous conditions currently exist within the enclosed space. For example, the sensor data may indicate dangerous levels of toxic gas, the presence of flammable gases within the enclosed space, or a low oxygen concentration within the enclosed space, depending upon the sensors installed within the enclosed space. As such, an application (e.g., the application 116) may make the determination of unsafe conditions within the enclosed space, and may provide an alarm or an alert to an operator device (e.g., the operator device 118) at 206.

Regardless of the current conditions of the enclosed space, the sensor data received at 202 may be added to a database stored within a cloud-based platform (e.g., the cloud-based platform 114) such as on a server or other remote computing device which additionally may run the application previously discussed. The database may store the new sensor data as well as a historical record of the sensor data which may date back to include a pre-defined window of time. The stored data may be used at 212 to generate a historical trend using the available data, or to update a previously generated historical trend with the new sensor data received. The historical trend may be visualized in one or more plots or formatted reports such that the frequency of events within the enclosed space, as well as the speed at which the hazardous conditions develop, may be illustrated. The generated visuals for the historical trend, along with the trend data itself, may be output to the operator device at 214 in order to provide insight to an operator regarding the historical status of the enclosed space and possible sources of hazardous events.

From the historical trends generated or updated at 212, further operational insight may be generated by the application at 216. The operational insight may involve a single enclosed space, or may include data from a plurality of enclosed spaces such that a network of enclosed spaces may be analyzed. The operational insight generated at 216 may flag particular enclosed spaces for maintenance, review, or quality control to prevent further hazardous events, based upon the historical trends seen in the individual enclosed spaces and compared to similar enclosed spaces. Further, overall heatmaps of geographical areas may be generated at 216 to provide visualization of present conditions within a network of enclosed spaces, or to show problem areas where hazardous events are more common. The operational insights developed at 216, including the flagging and heatmaps, may be displayed to an operator at the operator device at 218. The output may be directly sent to an operator device at 218, or may be presented in a business intelligence dashboard on the cloud-based platform which may then be accessed via the operator device.

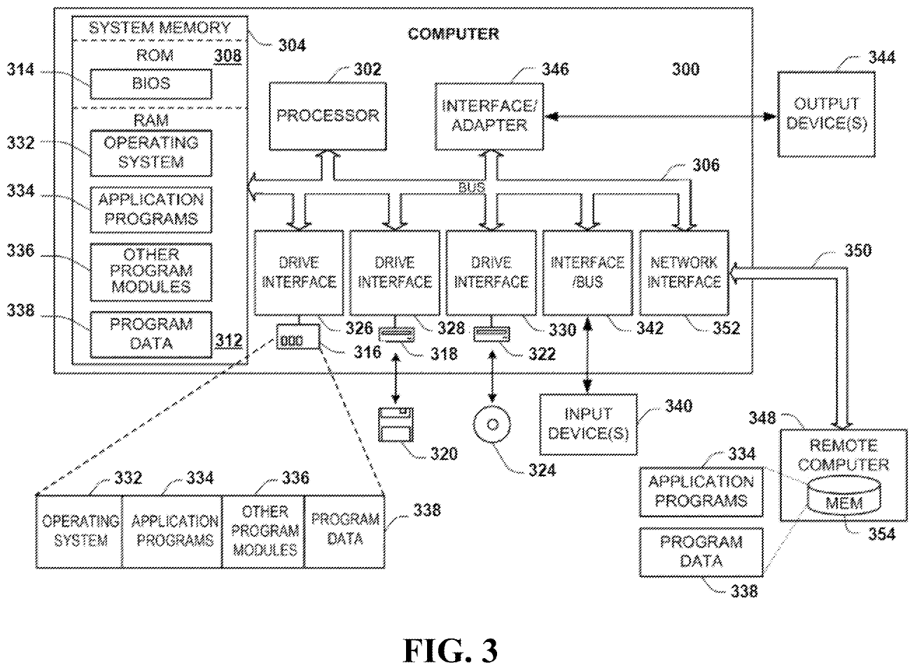

In view of the foregoing structural and functional description, those skilled in the art will appreciate that portions of the embodiments may be embodied as a method, data processing system, or computer program product. Accordingly, these portions of the present embodiments may take the form of an entirely hardware embodiment, an entirely software embodiment, or an embodiment combining software and hardware, such as shown and described with respect to the computer system of FIG. 3. Furthermore, portions of the embodiments may be a computer program product on a computer-usable storage medium having computer readable program code on the medium. Any non-transitory, tangible storage media possessing structure may be utilized including, but not limited to, static and dynamic storage devices, hard disks, optical storage devices, and magnetic storage devices, but excludes any medium that is not eligible for patent protection under 35 U.S.C. § 101 (such as a propagating electrical or electromagnetic signals per se). As an example and not by way of limitation, computer-readable storage media may include a semiconductor-based circuit or device or other IC (such, as for example, a field-programmable gate array (FPGA) or an ASIC), a hard disk, an HDD, a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a SECURE DIGITAL card, a SECURE DIGITAL drive, or another suitable computer-readable storage medium or a combination of two or more of these, where appropriate. A computer-readable non-transitory storage medium may be volatile, nonvolatile, or a combination of volatile and non-volatile, as appropriate.

Certain embodiments have also been described herein with reference to block illustrations of methods, systems, and computer program products. It will be understood that blocks and/or combinations of blocks in the illustrations, as well as methods or steps or acts or processes described herein, can be implemented by a computer program comprising a routine of set instructions stored in a machine-readable storage medium as described herein. These instructions may be provided to one or more processors of a general purpose computer, special purpose computer, or other programmable data processing apparatus (or a combination of devices and circuits) to produce a machine, such that the instructions of the machine, when executed by the processor, implement the functions specified in the block or blocks, or in the acts, steps, methods and processes described herein.

These processor-executable instructions may also be stored in computer-readable memory that can direct a computer or other programmable data processing apparatus to function in a particular manner, such that the instructions stored in the computer-readable memory result in an article of manufacture including instructions which implement the function specified. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

In this regard, FIG. 3 illustrates one example of a computer system 300 that can be employed to execute one or more embodiments of the present disclosure. Computer system 300 can be implemented on one or more general purpose networked computer systems, embedded computer systems, routers, switches, server devices, client devices, various intermediate devices/nodes or standalone computer systems. Additionally, computer system 300 can be implemented on various mobile clients such as, for example, a personal digital assistant (PDA), laptop computer, pager, and the like, provided it includes sufficient processing capabilities.

Computer system 300 includes processing unit 302, system memory 304, and system bus 306 that couples various system components, including the system memory 304, to processing unit 302. Dual microprocessors and other multi-processor architectures also can be used as processing unit 302. System bus 306 may be any of several types of bus structure including a memory bus or memory controller, a peripheral bus, and a local bus using any of a variety of bus architectures. System memory 304 includes read only memory (ROM) 310 and random access memory (RAM) 312. A basic input/output system (BIOS) 314 can reside in ROM 310 containing the basic routines that help to transfer information among elements within computer system 300.

Computer system 300 can include a hard disk drive 316, magnetic disk drive 318, e.g., to read from or write to removable disk 320, and an optical disk drive 322, e.g., for reading CD-ROM disk 324 or to read from or write to other optical media. Hard disk drive 316, magnetic disk drive 318, and optical disk drive 322 are connected to system bus 306 by a hard disk drive interface 326, a magnetic disk drive interface 328, and an optical drive interface 330, respectively. The drives and associated computer-readable media provide nonvolatile storage of data, data structures, and computer-executable instructions for computer system 300. Although the description of computer-readable media above refers to a hard disk, a removable magnetic disk and a CD, other types of media that are readable by a computer, such as magnetic cassettes, flash memory cards, digital video disks and the like, in a variety of forms, may also be used in the operating environment; further, any such media may contain computer-executable instructions for implementing one or more parts of embodiments shown and described herein.

A number of program modules may be stored in drives and RAM 310, including operating system 332, one or more application programs 334, other program modules 336, and program data 338. In some examples, the application programs 334 can include hazard determination modules, trend generation modules, or operational insight modules, such as those executed as part of the application 116, and the program data 338 can include sensor data, hazardous event flags, historical trendlines, timestamps, and generated operational insights. The application programs 334 and program data 338 can include functions and methods programmed to receive sensor data, determine conditions within an enclosed space, and output processed data or alerts to an operator or business intelligence dashboard, such as shown and described herein.

A user may enter commands and information into computer system 300 through one or more input devices 340, such as a pointing device (e.g., a mouse, touch screen), keyboard, microphone, joystick, game pad, scanner, and the like. For instance, the user can employ input device 340 to edit or modify provided operational insight or maintenance plans. These and other input devices 340 are often connected to processing unit 302 through a corresponding port interface 342 that is coupled to the system bus, but may be connected by other interfaces, such as a parallel port, serial port, or universal serial bus (USB). One or more output devices 344 (e.g., display, a monitor, printer, projector, or other type of displaying device) is also connected to system bus 306 via interface 346, such as a video adapter.

Computer system 300 may operate in a networked environment using logical connections to one or more remote computers, such as remote computer 348. Remote computer 348 may be a workstation, computer system, router, peer device, or other common network node, and typically includes many or all the elements described relative to computer system 300. The logical connections, schematically indicated at 350, can include a local area network (LAN) and/or a wide area network (WAN), or a combination of these, and can be in a cloud-type architecture, for example configured as private clouds, public clouds, hybrid clouds, and multi-clouds. When used in a LAN networking environment, computer system 300 can be connected to the local network through a network interface or adapter 352. When used in a WAN networking environment, computer system 300 can include a modem, or can be connected to a communications server on the LAN. The modem, which may be internal or external, can be connected to system bus 306 via an appropriate port interface. In a networked environment, application programs 334 or program data 338 depicted relative to computer system 300, or portions thereof, may be stored in a remote memory storage device 354. The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

The invention claimed is:

1. A system comprising:
a plurality of gas accumulation sensors each operable to detect a gas within an associated enclosed space;
a communications transceiver communicatively coupled to each gas accumulation sensor; and
a computer platform operable to execute a gas accumulation application,
wherein the gas accumulation application receives one or more readings from each gas accumulation sensor via a communicatively coupled communications transceiver, processes the one or more readings, and alerts an operator with a safety status of the enclosed space.

2. The system of claim 1, further comprising a LoRa gateway installed within range of the communications transceiver and configured to connect the plurality of gas accumulation sensors to an Internet of Things.

3. The system of claim 2, wherein the LoRa gateway is installed within an existing telecommunications tower.

4. The system of claim 1, wherein the plurality of gas accumulation sensors measure an accumulation of toxic gases, a presence of flammable gases, an oxygen content, or any combination thereof within the associated enclosed space.

5. The system of claim 4, wherein the plurality of gas accumulation sensors take measurements at a predefined sampling rate.

6. The system of claim 4, wherein the plurality of gas accumulation sensors provide measurements to the gas accumulation application via report-by-exception monitoring, wherein the gas accumulation application only receives measurements outside of a predefined operating range.

7. The system of claim 1, wherein the plurality of gas accumulation sensors are installed at a lower end of the associated enclosed space.

8. The system of claim 7, wherein the plurality of gas accumulation sensors are installed within about 0.5 meters of a bottom of the enclosed space.

9. A method comprising:
receiving one or more measurements relating to gas in one or more enclosed spaces from sensors that are each associated with an enclosed space;
determining a status of each enclosed space including whether hazardous conditions exist from the one or more measurements; and
alerting an operator to the status of each enclosed space,
wherein the one or more measurements comprise a level of accumulated toxic gas, a presence of flammable gas, an oxygen content reading, or any combination thereof.

10. The method of claim 9, further comprising:
storing the one or more measurements relating to gas with historical measurement data from the sensors;
generating historical trends of the one or more measurements over time; and
outputting the historical trends to the operator,
wherein the historical trends identify a frequency and duration of hazardous events within the one or more enclosed spaces.

11. The method of claim 10, further comprising:
generating operational insight from the historical trends involving each enclosed space, or a network incorporating a plurality of the one or more enclosed spaces; and
presenting the operational insight to the operator,
wherein the operational insight comprises one or more flags for the one or more enclosed spaces for maintenance, review, quality control, or any combination thereof, heat maps of hazard areas within the one or more enclosed spaces or within the network, or any combination thereof.

12. The method of claim 11, wherein an alert to the operator, the historical trends, the operational insight, or any combination thereof is presented within a business intelligence dashboard on a cloud-based platform.

13. The method of claim 12, wherein the business intelligence dashboard further presents a real-time status of the one or more measurements.

14. The method of claim 9, wherein the enclosed space is a telecommunications maintenance hole.

15. The method of claim 9, further comprising sending one or more measurements from the sensors via a LoRa network.
